(19) United States
(12) Patent Application Publication
(10) Pub. No.: US 2015/0141748 A1
(43) Pub. Date: May 21, 2015

(54) Sexual Aid Device

(71) Applicant: Sean Campbell, Laurelton, NY (US)

(72) Inventor: Sean Campbell, Laurelton, NY (US)

(21) Appl. No.: 14/085,330

(22) Filed: Nov. 20, 2013

(51) Int. Cl. A61H 19/00; A61F 5/41

(52) U.S. Cl. CPC A61H 19/50; A61F 5/41; A61H 19/44; A61H 19/32; A61F 2005/417
USPC 600/38

(57) ABSTRACT

A sexual aid operable to be surroundably mounted around the penis wherein the sexual aid is operable to produce vibrations. The sexual aid further includes a body that is generally cylindrical in shape being substantially hollow and having a passage therethrough. The body is formed from a first layer and a second layer. Intermediate the first layer and the second layer in pockets are a plurality of vibration members. A controller is present and operably_coupled to the plurality of vibration members. The sexual aid is transitioned from a first mode to a second mode via the controller wherein in the second mode the sexual aid is operable to produce vibrations.

### FIELD OF THE INVENTION

The present invention relates to a sexual aid device, more specifically but not by way of limitation, a device that is circumferentially mounted to the male sex organ for use during intercourse that includes a plurality of vibratory members that provide a vibratory sensation during sex practices.

### BACKGROUND

Sexual aid devices have been used during the practice of sexual activities for centuries. Devices such as but not limited to artificial phalluses have been used in ceremonies ranging from ancient cultural rituals to the practice of marital proceedings. In addition, a variety of sexual aid devices have been used for the simple purpose of pleasure by couples who are looking to enhance their sexual experience between each other. Furthermore, sexual aid devices such as but not limited to vibrators have been used as tools by sex counselors during the treatment and/or counseling of their patients.

Currently vibratory devices are mass produced in a variety of different styles such as but not limited to a conventional vibrator. These devices are often used by females that are no longer engaged in a sexual relationship with a partner or are used to assist in their stimulation during a sexual encounter with their partner. One problem with conventional vibrators is there conventional phallic shape does not allow participation by a male partner during use by a female. Typically the female will use the phallic shaped conventional vibrator to penetrate the orifice of her vagina in order to derive pleasure from the vibratory device. This can leave the male partner at least partially excluded from the sexual experience and result in difficulties in the relationship between the two individuals. Conventional vibrators also commonly referred to as dildos, can often become the females preferred method of sexual pleasure which can create further removal of the male from the intimacy of the sexual experience and ultimately the relationship. Another problem with conventional vibrators or dildos is that do not allow the male participating in the sexual experience to penetrate the orifice of the vagina as the conventional vibrator or dildo does not permit this act.

Accordingly there is a need for a vibratory device that can be used by the male during a sexual experience with a female that will provide a vibratory sensation to the female while allowing the male to penetrate the orifice of the vagina with his sexual organ.

### SUMMARY OF THE INVENTION

It is the object of the present invention to provide a vibratory sexual aid that is circumferentially mounted to the male sex organ so as to allow the male sex organ to penetrate the orifice of the vagina and simultaneously provide a vibration sensation to a female partner.

Another object of the present invention is to provide a vibratory sexual aid that is surroundably mounted to the penis and that is generally cylindrical in shape having a first and second opening at opposing ends that are approximately equal in diameter.

A further object of the present invention is to provide a vibratory device that is surroundably mounted around substantially the length of the penis that has a first layer and a second layer.

An additional object of the present invention is to provide a vibratory sexual aid device that has at least one vibration member intermediate the first layer and the second layer.

Still another object of the present invention is to provide a vibratory sexual aid device that has at least one vibration member that is generally elongated in shape.

Yet a further object of the present invention is to provide a vibratory sexual aid device that includes an integrated power supply.

Still another object of the present invention is to provide a vibratory sexual aid device that can be worn underneath a male prophylactic.

An additional object of the present invention is to provide a vibratory sexual aid device that has at least one vibration member that is positioned to stimulate a female erogenous zone.

A further object of the present invention is to provide a vibratory sexual aid device that has at least one vibratory device that can be programmed to vibrate at different frequencies.

To the accomplishment of the above and related objects the present invention may be embodied in the form illustrated in the accompanying drawings. Attention is called to the fact that the drawings are illustrative only. Variations are contemplated as being a part of the present invention, limited only by the scope of the claims.

### BRIEF DESCRIPTION OF THE DRAWINGS

A more complete understanding of the present invention may be had by reference to the following Detailed Description and appended claims when taken in conjunction with the accompanying Drawings wherein:

FIG. 1 is a perspective view of an embodiment of the present invention; and

FIG. 2 is a cross-sectional view of the embodiment of the present invention illustrated in FIG. 1; and

FIG. 3 is a cross-sectional diagram of the vibratory member of the present invention; and

FIG. 4 is a perspective view of an alternative embodiment of the present invention.

### DETAILED DESCRIPTION

Referring now to the drawings submitted herewith, wherein various elements depicted therein are not necessarily drawn to scale and wherein through the views and figures like elements are referenced with identical reference numerals, there is illustrated a sexual aid 100 constructed according to the principles of the present invention.

Referring in particular to FIG. 1, there is a sexual aid 100 illustrated therein that further includes a body 10. The body 10 is generally cylindrical in shape having a first end 11 and a second end 12 and is manufactured from a resilient expandable material such as but not limited to silicon rubber. The body 10 further includes a first opening 15 proximate the first end 11 and a second opening 16 proximate the second end 12. The body 10 is manufactured so as to surroundably the shaft of the male penis. The body 10 has a diameter that is at least smaller than the diameter an average erect male penis. The body 10 is configured to be diametrically expandable so as to accommodate a penis therethrough and retract to bias thereagainst. The body 10 is stretched to accommodate a penis wherein the penis is journaled through the first opening 15 and extends through the passage 63 and propagates through the second opening 16. As the body 10 is stretched to accommodate an erect penis therein the body 10 will retract to its first non-expanded position. As the body 10 attempts to retract to its original non-biased position, the body 10 is biased against and generally towards the shaft of the erect penis disposed therein. Subsequent the user surroundably mounting the body 10 to the shaft of an erect penis, the body 10 is biased thereagainst with sufficient force so as to maintain the position of the body 10 on the erect penis so as to substantially maintain the body 10 on the penis during sexual activities such as but not limited to sexual intercourse. It is additionally contemplated within the scope of the present invention that the body 10 could be releasably secured to an artificial penis such as but not limited to a dildo. In this embodiment, a dildo or similar device is provided with the sexual aid 100 and the body 10 is releasably secured to a dildo in a similar manner as described herein with a penis. It is further contemplated within the scope of the present invention that the body 10 could be integrally secured to an artificial shaft resembling a male penis. It is contemplated within the scope of the present invention that the body 10 could be manufactured in numerous different diameters so as to accommodate a variety of different penis sizes and in order to achieve the desired functionality as described herein. Furthermore, it is additionally contemplated within the scope of the present invention that the body 10 could be manufactured in numerous different lengths so as to accommodate a variety of different size penis'. While not illustrated herein, it is contemplated within the scope of the present invention that the body 10 could have secured proximate the first end a strap or similar device designed to mount to the testicles or around the user's waist so as to provide assistance in maintaining the position of the body 10 around the user's penis. Additionally, it is further contemplated within the scope of the present invention that the body 10 could be manufactured from a semi-rigid material so as to allow men suffering from impotence to utilize the device without the need for establishing and/or maintaining an erect penis.

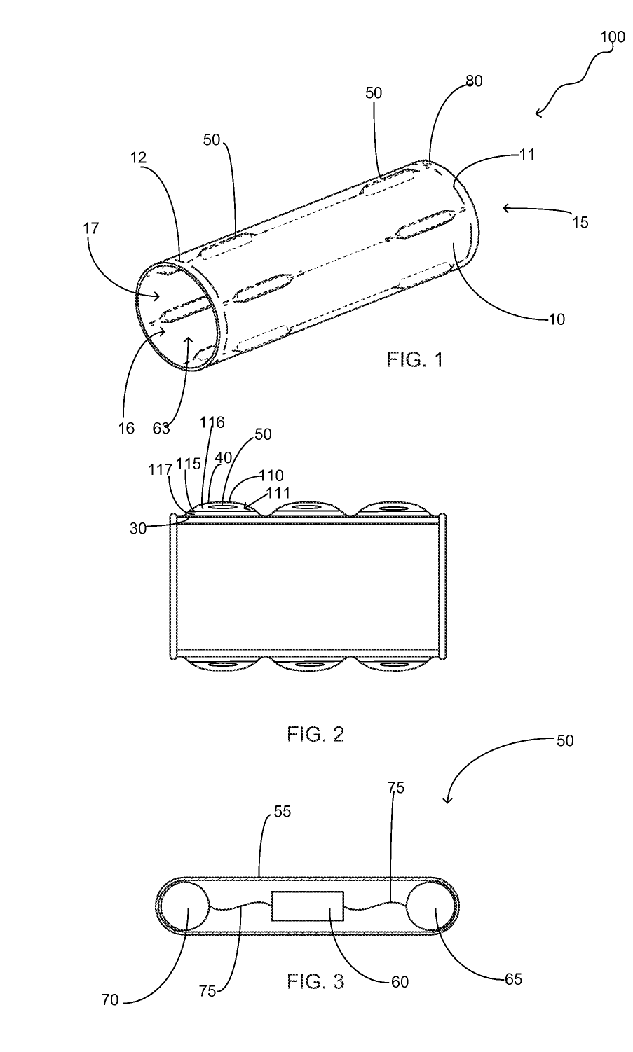

As shown in particular in FIG. 2, the body 10 further includes a first layer 30 and a second layer 40. The first layer 30 and the second layer 40 are approximately equivalent in size and secured utilizing a suitable durable method such as but not limited to chemical adhesion. The second layer 40 is secured utilizing suitable durable techniques to the first layer 30 so as to form pockets 110. The pockets 110 function to house the vibration member 50. The pockets 110 further include membrane 115 that is suspended across the interior volume 111 of the pocket 110. The membrane 115 divides the pocket 110 into a first portion 116 and a second portion 117. The vibration member 50 is disposed within the first portion 116 of the pocket 110. This positioning places the vibration member 50 such that the vibrations generated therefrom are transferred to the partner of a user of the sexual aid 100 as only the second layer 40 is intermediate the partner of the sexual aid 100 and the vibration member 50. The second portion 117 of the pocket 110 further contains a gel. The gel is operable to inhibit the transfer of vibrations from the vibration member 50 to the user of the sexual aid 100. While numerous types gels could be utilized to fill the second portion 117 of the pocket 110, good results have been achieved utilizing a silicone gel.

The sexual aid 100 as presented herein is designed to provide vibrational stimulation to the partner of the user of the sexual aid 100 while substantially inhibiting the feel of the vibrations by the user. This is accomplished through disposing the vibration members 50 within the pockets 110 and further providing the gel-filled second portion 117 of the pocket 110. The membrane 115 is manufactured from a suitable durable material such as but not limited to plastic film or rubber and is secured within the pocket 110 utilizing suitable durable techniques. The first layer 30 is manufactured from a visco-elastic vibration reducing material such as but not limited to amorphous polymers. The combination of the material of the first layer 30 and the gel-filled second portion 117 functions to substantially inhibit the transmission of vibrations to the penis of the user of the sexual aid 100. While many females experience pleasure from vibratory stimulation to the clitoris, most males experience discomfort from vibration to their penis. The sexual aid 100 as constructed addresses this issue. While a membrane 115 is illustrated and discussed herein, it is contemplated within the scope of the present invention that the pocket 110 could be constructed without the membrane 115 and the entire pocket be filled with a gel. The first layer 30 is relatively thin in thickness in order to facilitate easy application of the sexual aid 100. While in its preferred embodiment the first layer 30 is relatively thin in construction, it is contemplated within the scope of the present invention that the first layer 30 could be manufactured such that the first layer 30 is at least one-quarter inch in thickness. This substantially thicker first layer 30 would be operable to increase the perceived width of the erect penis of the user disposed within the sexual aid 100.

The vibration members 50 include a body 55 that is generally rod-shaped having a first end 51 and a second end 52 that are generally rounded. The vibration members 50 additionally create an area of extended height of the exterior surface 71 that greater in height that that of the exterior surface 91 that does not have a vibration member 50 proximate thereto. The first end 51 and second end 52 are generally rounded in shape so as to provide a more comfortable feel for the recipient of the penis having the sexual aid 100 surroundably mounted thereto. Those skilled in the art will recognize that the first end 51 and second end 52 could be formed in numerous different shapes and still achieve the desired functionality as described herein. Additionally, it is contemplated within the scope of the present invention that the body 55 of the vibration members 50 could be manufactured in numerous different shapes and sizes. The body 55 of the vibration members 50 is manufactured from a suitable durable material such as but not limited to plastic. While no particular length of the vibration member 50 is required, good results have been achieved utilizing a vibration member 50 that is approximately one inch in length. As illustrated herein in FIGS. 1 and 2 the vibration members 50 are mounted in a generally linear manner along the length of the body 10. Additionally, the vibration members 50 are mounted around the diameter of the body 10 at approximately equivalent intervals. Although the embodiment illustrated in FIGS. 1 and 2 illustrate the vibration members 50 oriented in a linear manner disposed around the diameter in an approximately equivalent intervals, it is contemplated within the scope of the present invention that the vibration members 50 could be mounted in numerous different patterns.

The vibration members 50 further include a power supply 60 and a first vibration motor 65 and a second vibration motor 70. FIG. 3 illustrates a diagrammatic view of the vibration members 50. The power supply 60 is a conventional DC power supply such as but not limited to a lithium ion battery that is operably coupled to the first vibration motor 65 and the second vibration motor 70 via a conventional wire 75. The first vibration motor 65 and the second vibration motor 70 are conventional vibration motors. As is known in the art, vibration motors are typically manufactured wherein the motor includes a rotational shaft having an offset counterweight mounted to the end of the shaft. As the motor spins the shaft the offset counterweight causes a vibration that is then transferred through the housing of the vibration motor. While the vibration members 50 are illustrated herein as having a first vibration motor 65 and a second vibration motor 70, it is contemplated within the scope of the present invention that the vibration members 50 could have as few as one vibration motor or a plurality thereof. Furthermore, it is contemplated within the scope of the present invention that the vibration members 50 could be arranged on the body 10 in numerous different patterns. More specifically but not by way of limitation, while FIG. 1 and FIG. 2 illustrate herein vibration members 50 that are disposed within the body 10 in a linear manner and disposed at approximately equivalent intervals around the circumference of the body 10, it is additionally contemplated within the scope of the present invention that the vibration members 50 could be disposed in any quantity and any pattern within the body 10.

The vibration members 50 are operably coupled to a controller 80. The controller 80 is secured to the body 10 proximate the first end 11 utilizing suitable durable methods such as but not limited to chemical adhesion. The controller 80 contains a conventional integrated circuit board that contains all of the electronics necessary to receive, store and manipulate data so as to provide operational control of the sexual aid 100. The controller 80 functions to activate the vibration members 80 sending a signal to each vibration member 50 activating the power supply 60 therein. While not illustrated herein, it is contemplated within the scope of the present invention that the controller 80 includes a switch that when depressed will either close or open the electrical circuit between the controller 80 and the vibration members 50. The controller 80 further provides control to determine the quantity of vibration members 50 that are activated. More specifically but not by way of limitation, the controller 80 function to activate at least a portion or substantially all of the vibration members 50 subsequent activation. It is contemplated within the scope of the present invention that the controller 80 cold be programmed to activate only a portion of the vibration members 50 disposed within the body 10. Furthermore, it is additionally contemplated within the scope of the present invention that the controller 80 cold be programmed so as to activate the vibration members 50 in a random pattern wherein only a portion of the vibration members 50 are producing vibrations and subsequently an alternative portion of vibration members 50 are activated to produce vibrations.

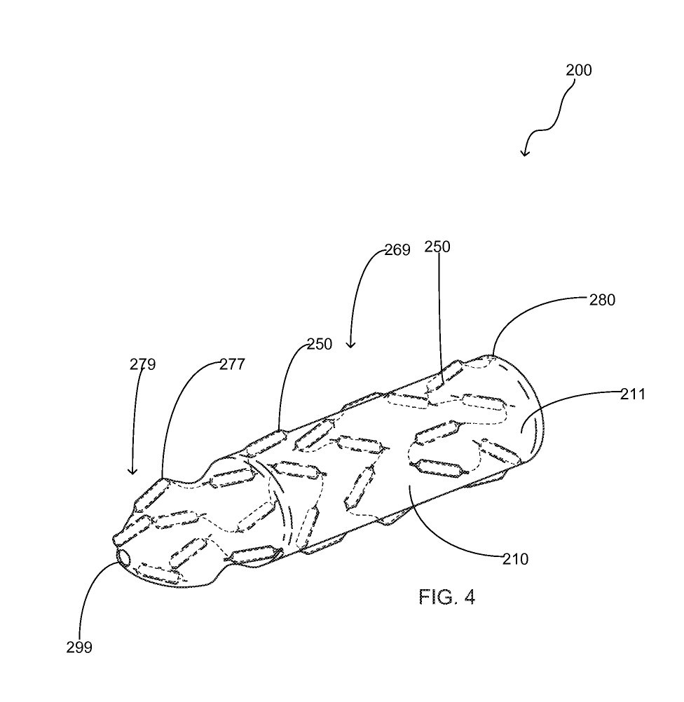

Referring in particular to FIG. 4, an alternative embodiment of the sexual aid 200 is illustrated therein. The sexual aid 200 further includes a body 210 that is manufactured in accordance with the first embodiment of the sexual aid 100 as described herein. The body 210 further includes a first portion 269 that is substantially cylindrical in shape and a second portion 279 that is integrally formed with the first portion 269 distal to the first end 211. The second portion 279 is formed to be of similar size and shape so as to accommodate the head of the penis. The second portion 279 further includes an aperture 299 so as to allow semen to egress therefrom during use. While the sexual aid 200 is illustrated herein as having aperture 299, it is contemplated within the scope of the present invention that the sexual aid 200 could be manufactured without an aperture 299. The vibration members 250 are disposed within the body 210 enclosed in pockets 277, as discussed herein for the preferred embodiment, in a random pattern and are operably controlled by the controller 280. The vibration members 250 are constructed in the same manner as the vibration members 50. It is contemplated within the scope of the present invention that the controller 280 of the alternative embodiment of the sexual aid 200 is constructed in the same manner and contains all of functionality as described herein of the controller 80 of the sexual aid 100.

While not illustrated herein, it is contemplated within the scope of the present invention that the sexual aid 100 and the sexual aid 200 could further include a plurality of lights embedded therein.

Referring in particular to FIG. 1, a description of the operation of the sexual aid 100 is as follows. In use, the user will expand the diameter of the body 10 so as to journal the user's penis therethrough. Subsequent the user having journaled the penis through the passage 17, the body 10 being constructed of a resilient material will attempt to return to its original unbiased position. As the diameter of the passage 17 is smaller than the diameter of the shaft of the average male penis, the body 10 is biased against the shaft of the penis so as to substantially maintain the position of the sexual aid during use. Ensuing the surroundable mounting of the body 10 to the penis of the user, the user will engage the controller 80 so as to activate the vibration members 50. The vibration members 80 are activated in a plurality of patterns as discussed herein. Following use of the sexual aid 100, the user will remove the sexual aid from the shaft of their penis.

In the preceding detailed description, reference has been made to the accompanying drawings that form a part hereof, and in which are shown by way of illustration specific embodiments in which the invention may be practiced. These embodiments, and certain variants thereof, have been described in sufficient detail to enable those skilled in the art to practice the invention. It is to be understood that other suitable embodiments may be utilized and that logical changes may be made without departing from the spirit or scope of the invention. The description may omit certain information known to those skilled in the art. The preceding detailed description is, therefore, not intended to be limited to the specific forms set forth herein, but on the contrary, it is intended to cover such alternatives, modifications, and equivalents, as can be reasonably included within the spirit and scope of the appended claims.

What is claimed is:

1. A sexual aid comprising:
a body, said body being generally cylindrical in shape, said body being hollow and having a passage therethrough, said body further being configured to be diametrically expandable, said body having an exterior surface, said body configured to have journaled therethrough a support structure, said body being manufactured from a first layer and a second layer, said first layer and said second layer being secured so as to form pockets, said pockets having an interior volume, said first layer being adjacent the support structure; and
at least one vibration member, said at least one vibration member being disposed within said pocket, said at least one vibration member having a first end and a second end, said at least one vibration member operable to produce vibrations, said at least one vibration member having a first vibration motor and a second vibration motor.

2. The sexual aid as recited in claim 1, wherein said support structure is selected from a group consisting of at least one of the following: a human penis or a dildo.

3. The sexual aid as recited in claim 2, wherein said at least one vibration member further includes a vibration motor, said vibration motor disposed within said at least one vibration member, said vibration motor being proximate said first end of said at least one vibration member.

4. The sexual aid as recited in claim 3, wherein said pocket further includes a membrane, said membrane suspended across the interior volume of said pocket so as to divide the interior volume of said pocket into a first portion and a second portion.

5. The sexual aid as recited in claim 4, wherein said at least one vibration member is disposed within said first portion of said pocket.

6. The sexual aid as recited in claim 5, wherein said second portion of said pocket is filled with a silicone gel.

7. The sexual aid as recited in claim 6, wherein said first layer of said body is manufactured from an amorphous polymer so as to reduce vibration transmission to the penis.

8. A sexual aid configured to be engaged with a support structure comprising:
a body, said body being generally cylindrical in shape, said body being hollow and having a passage therethrough, said body having a first end and a second end, said body having a first opening proximate said first end, said body having a second opening proximate said second end, said body configured to be diametrically expandable, said body having a first layer and a second layer, said first layer and said second layer being joined so as to form a plurality of pockets therebetween, said plurality of pockets having an interior volume, said first layer being adjacent to the support structure;
a plurality of vibration members, said plurality of vibration members being disposed within said interior volume of said plurality of pockets, said plurality of vibration members being elongated in shape, said plurality of vibration members having a first end and a second end, said plurality of vibration members having a first mode and a second mode, said plurality of vibration members having a first vibration motor and a second vibration motor disposed therein; and
wherein said plurality of vibration members are operable to produce vibrations in said second mode.

9. The sexual aid as recited in claim 8, wherein said plurality of pockets further include a membrane, said membrane being mounted across said interior volume so as to divide said interior volume into a first portion and a second portion.

10. The sexual aid as recited in claim 9, and further including a controller, said controller intermediate said first layer and said second layer of said body, said controller being proximate said first end, said controller operably coupled to said plurality of vibration members, said controller operable to transition said plurality of vibration members from said first mode to said second mode.

11. The sexual aid as recited in claim 10, wherein said plurality of vibration members are disposed within said first portion of said plurality of pockets.

12. The sexual aid as recited in claim 10, wherein said support structure is selected from a group consisting of at least one of the following: a human penis or a dildo.

13. The sexual aid as recited in claim 12, wherein said second portion of said plurality of pockets are filled with a silicon gel.

14. The sexual aid as recited in claim 13, wherein said opening proximate said first end and said opening proximate said second end are approximately the same diameter.

15. A sexual aid operable to substantially cover a penis comprising:
a body, said body having a hollow passage therethrough, said body having a first layer and a second layer, said first layer and said second layer being integrally formed so as to create a plurality of pockets therebetween, said plurality of pockets having an interior volume, said plurality of pockets further including a membrane therein operable to divide said interior volume into a first portion and a second portion, said second portion of said plurality of pockets being adjacent said first layer, said first layer being adjacent to the penis, said first layer being configured to substantially inhibit vibrations from being transmitted to the penis, said body configured to be diametrically expandable;
a plurality of vibration members, said plurality of vibration members being disposed within said first portion of said plurality of pockets, said plurality of vibration members being elongated in shape, said plurality of vibration members having a first end and a second end, said plurality of vibration members having a first vibration motor disposed therein proximate said first end of said plurality of vibration members, said plurality of vibration members having a second vibration motor disposed therein proximate said second end of said plurality of vibration members, said plurality of vibration members being intermediate said first layer and said second layer of said body, said first end and said second end of said plurality of vibration members being generally rounded in shape, said plurality of vibration members having a first mode and a second mode; and
wherein said plurality of vibration members are operable to produce vibrations in said second mode.

16. The sexual aid as recited in claim 15, and further including a controller, said controller being proximate said first end of said body, said controller intermediate said first layer and said second layer of said body, said controller being proximate said first end, said controller operably coupled to said plurality of vibration members, said controller operable to transition said plurality of vibration members from said first mode to said second mode.

17. The sexual aid as recited in claim 12, wherein said second portion of said plurality of pockets are filled with a silicon gel.

18. The sexual aid as recited in claim 17, wherein said plurality of vibration members further include a power supply, said power supply being intermediate said first vibration motor and said second vibration motor, said power supply operable to provide power to said first vibration motor and said second vibration motor.

19. The sexual aid as recited in claim 18, wherein said plurality of vibration members are secured within said plurality of pockets wherein the plurality of pockets are arranged in a random pattern.

20. The sexual aid as recited in claim 19, wherein said first layer is manufactured from an amorphous polymer so as to substantially reduce vibrations transmitted to the penis.
